(19) United States
(12) Patent Application Publication
(10) Pub. No.: US 2006/0260003 A1
(43) Pub. Date: Nov. 16, 2006

(54) Device and Method to Facilitate Directed Delivery and Electroporation

(75) Inventors: Richard Gilbert, Tampa, FL (US); Mark J. Jaroszeski, Tampa, FL (US); Richard Heller, Temple Terrace, FL (US)

Correspondence Address: SMITH HOPEN, PA, 180 PINE AVENUE NORTH, OLDSMAR, FL, 34677, US

(73) Assignee: University of South Florida, Tampa, FL (US)

(21) Appl. No.: 11/306,945

(22) Filed: Jan. 17, 2006

Related U.S. Application Data
(63) Continuation of application No. PCT/US04/23052, filed on Jul. 16, 2004.
(60) Provisional application No. 60/481,096, filed on Jul. 16, 2003.

(51) Int. Cl. C12M 1/42; A01H 1/00

(52) U.S. Cl. 800/278; 435/285.2

(57) ABSTRACT

The present invention provides an apparatus and method that will accomplish molecular delivery and subsequent electroporation with a single apparatus without the need for separate molecule injection and placement of the electroporation applicators.

### CROSS REFERENCE TO RELATED APPLICATIONS

This application is a continuation of PCT Application No. PCT/US2004/23052, filed Jul. 16, 2004, which claims benefit of U.S. Provisional Application No. 60/481,096, filed Jul. 16, 2003

### BACKGROUND OF INVENTION

There are various electroporation applicators and methods of use known in the art. The prior art methods involve two independent procedures, the introduction of a target molecule into a target tissue and the subsequent electroporation of the target tissue to induce electroporation. The prior art methods require two separate devices, one to introduce the target molecule and a second to provide for the electroporation of the target tissue site.

Prior art methods to introduce a target molecule into a target tissue include the use of intrusive instruments or the application of an electric field in direct contact with the skin with the stated purpose of altering the skin to allow a drug to move through the external skin barrier.

Prior art methods and apparatus insert the target molecule with techniques described above and then require placement of the electroporation applicators. As such, there is no guarantee that the injected target molecule is still within the tissue area to be electroporated. The molecule could have moved out of the area by the time the electroporation applicators are in place. This deficiency in the prior art techniques is even more pronounced in molecular treatments requiring multiple target molecules and multiple electroporation protocols. The treatment using the techniques known in the art quickly becomes extremely labor and time intensive.

Accordingly, there is a need in the art for a method and apparatus to facilitate the directed delivery and subsequent electroporation of a target molecule in-vivo without the need for a separate molecule injection system and subsequent placement of the electroporation applicators.

### SUMMARY OF INVENTION

The present invention provides a method and apparatus to facilitate the directed delivery and subsequent electroporation of a target molecule in-vivo.

An apparatus in accordance with the present invention includes a reservoir having an electrically conductive top end cap and a micro-porous bottom end cap. The bottom end cap further includes at least one electroporation applicator.

To electrically isolate the electrically conductive top end cap from the side walls of the reservoir, the apparatus includes an electrical insulator positioned between the underside of the top cap edge and the exterior side wall of the reservoir. As such, when a voltage from a polarization supply voltage in circuit communication with the top end cap is applied, the reservoir is electrically isolated from the top end cap.

The bottom end cap of the reservoir is micro-porous and may include a micro-porous membrane. Additionally, at least one electroporation applicator is embedded in the micro-porous membrane or otherwise secured to the bottom end cap of the reservoir. Accordingly, the bottom end cap is adapted to be positioned in contact with a target tissue and the electroporation applicators are non-penetrating in nature.

It is within the scope of the present invention to have a plurality of electroporation applicators embedded in the micro-porous membrane of the bottom end cap. Each of the plurality of electroporation applicators are in circuit communication with an electroporation supply voltage.

The reservoir of the present invention is adapted to contain a charged entity, or target molecule, to be transported and subsequently electroporated into the target tissue. As such, the reservoir may be non-conductive and fluid tight as required. A variety of target molecules are envisioned by the present invention, including a protein, a plasmid and a therapeutic drug.

In accordance with an additional embodiment of the invention, the apparatus further includes a plurality of isolated compartments making up the reservoir. Each compartment is isolated from the others, while still maintaining continuity with the top cap and bottom cap of the reservoir. Accordingly, different charged entities may be positioned within each of the individual compartments. With this embodiment, the top end cap of the reservoir further includes a plurality of electrically isolated portions, each electrically isolated portion of the end cap associated with each of the plurality of isolated compartments.

The present invention provides a method for molecular delivery and electroporation, the method includes containing a target molecule or charged entity within a reservoir, contacting a micro-porous bottom end cap of the reservoir to a surface of a cellular tissue, applying a polarization voltage to a conductive top end cap of the reservoir adapted to transport the target molecule through the micro-porous bottom end cap of the reservoir and diffuse the target molecule across the surface of the cellular tissue, and applying an electroporation voltage to at least one electroporation applicator positioned on the micro-porous bottom end cap to introduce the target molecule into the interior of a cell of the cellular tissue.

In an additional embodiment, applying the polarization voltage further includes applying a polarization voltage following a predetermined protocol. The predetermined protocol species the voltage levels and durations of application, thereby providing increased control over the transport of the target molecule and diffusion into the target tissue. The application of the polarization voltage is effective in altering the electrochemical potential of the target molecule. The electroporation voltage applied to the electroporation applicators may also be controlled by a similar predetermined protocol.

In an additional embodiment, an independent polarization voltage is applied to a plurality of portions of the top cap, thereby affecting the transport of molecules contained within each of a plurality of isolated compartments of the reservoir.

The apparatus and method in accordance with the present invention to facilitate the directed delivery and electroporation of a target molecule relative to a target tissue provide many advantages over the known prior art.

Because the diffusive delivery mechanism and the electroporation mechanism are contained within a single device a more efficient and effective method is provided. By polarizing the top end cap of the reservoir, the molecules of interest are diffused into the tissue. The application of an electroporation voltage then induces electroporation within the same area of tissue. It is not necessary to remove the means for transporting the molecule across the tissue and then align the electroporation applicators because these two features are combined in this invention.

The apparatus overcomes the problem in the prior art of insuring that the application of the electroporation treatment is taking place at the same location in the tissue where the molecule was inserted. Due to the time delay caused by the additional step in the prior of placing the electroporation applicators, the injected molecule may have moved from the injection site and therefore the effectiveness of the electroporation effect is greatly reduced. Since the electroporation applicators are an integral component of the present invention, the delivered molecules are assured to be within the electroporation applicators electric field of influence during the treatment procedure.

An additional benefit of the present invention is the ability to apply multiple treatments to the same area of tissue without the need to reposition the electroporation applicators of the transport mechanism. Additionally, the isolated compartments of the reservoir provide for a complex protocol treatment utilizing a single device. The use of a control protocol for the application of the polarizing and electroporation voltages will allow repeat molecule application to a tissue site, followed by an electroporation event, without requiring additional intervention by a physician. Additionally, the invention provides for a treatment protocol combining early and late gene expression. Control over the electroporation parameters and the ability to combine two or more effective agents in the reservoir allows for the use of the device in broader gene therapy applications where two different expression proteins, as the same tissue site, at prescribed times, would prove valuable.

The electroporation applicators of the present invention are non-intrusive, therefore the method does not discomfort the patent.

The present invention provides an apparatus and method that will accomplish molecular delivery and subsequent electroporation with a single apparatus without the need for separate molecule injection and placement of the electroporation applicators.

### BRIEF DESCRIPTION OF THE DRAWINGS

For a fuller understanding of the nature and objects of the invention, reference should be made to the following detailed description, taken in connection with the accompanying drawings, in which:

FIG. 1 is an illustrative side view of the apparatus in accordance with the present invention.

FIG. 2 is an illustrative side view of the apparatus in accordance with the present invention, having isolated compartments within the reservoir.

### DETAILED DESCRIPTION OF THE PREFERRED EMBODIMENT

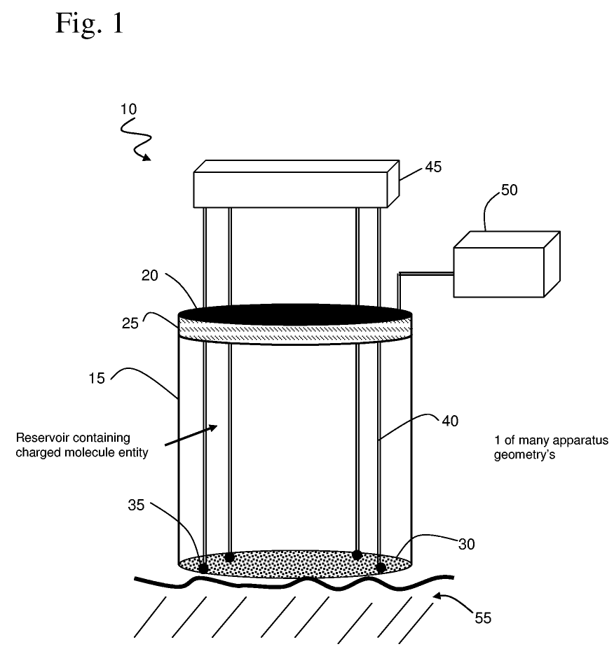

With reference to FIG. 1, in one embodiment, the invention 10 includes of a top end cap 20, a volumetric containment vessel or reservoir 15 and a bottom end cap 30. The top end cap 20 is constructed of a material with the appropriate dielectric properties to assure that it will hold a charge sufficient to polarize charged entities contained within the volumetric vessel 15. Accordingly, the top end cap could be a stainless steel frit or fabricated from a variety of other semi-conductive materials known in the art. Materials with such dielectric properties that can be machined or molded to the required top end cap 20 are readily available in the art. The reservoir 15 is preferably fabricated from a non-conductive material that is impervious to the change entity or target molecule being stored. As such, the reservoir may be fabricated of a polymeric or a variety of other nonconductive materials known in the art. The bottom end cap of the reservoir 30 is fabricated of a micro-porous material or micro-porous membrane. Appropriate materials for the reservoir walls 15, the micro-porous membrane 30 and the conductive top end cap 20 are commercially available and known in the art.

To insulate the conductive top end cap 20 from the reservoir 15, an insulative seal 25 is positioned between the top end cap and the reservoir.

Polarization voltages are delivered to the top end cap 20 by a polarization voltage supply 50. Various means are envisioned to provide the electrical connection from the supply to the cap, including an electrical polarization tab integral to the surface of the top end cap.

The bottom end cap 30 further includes a plurality of electroporation applicators 35. These electroporation applicators may be embedded within the micro-porous material of the end cap 30, or otherwise secured by means known in the art. Each of the electroporation applicators 35 is in circuit communication with a source of electroporation voltage 45 through a plurality of conductive leads 40. The source 45 is capable of providing a variety of voltage application protocols. Additionally, the electroporation applicators 35 may be driven independently or in combination, for the application of the electroporation treatment as necessary by the particular application of the device. The conductive leads 40 connecting the electroporation applicators 35 to the source 45 may pass through the top end lid 20. Accordingly, the top end cap 20 may include electrical through-puts to insulate the leads 40 from the conductive top cap 20.

In an additional embodiment, the present invention provides a wide degree of freedom in the selection and control of the applied potential 50 that provides the polarization of the top end cap 20. This polarization voltage value controls the diffusion of the charged molecule through the apparatus and the tissue 55. Metered control of the molecule will allow effective electroporation based on predictable concentrations of the molecules at the electroporation target.

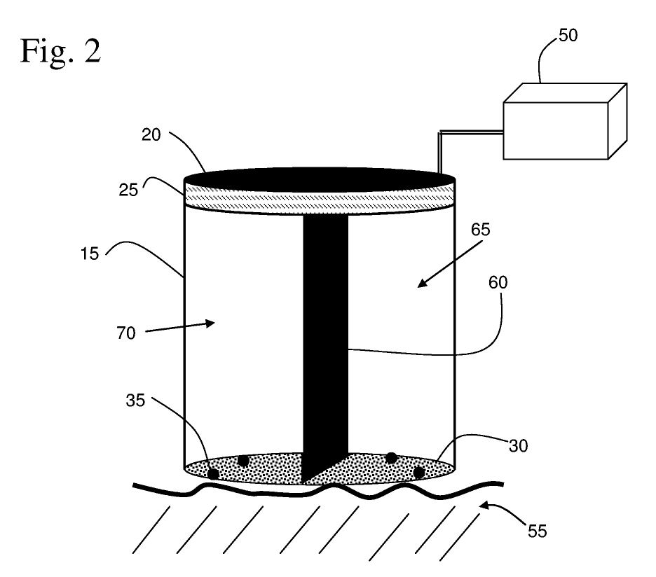

In an additional embodiment, with reference to FIG. 2, isolated compartments 65 and 70 are placed within the reservoir 15 that are effective in isolating different types of molecules within the reservoir 15. A non-porous barrier 60 is positioned or fabricated within the reservoir 15 to establish the isolation of the target molecules. While the figure illustrates two compartments, any number of isolated compartment are within the scope of the invention. With this embodiment, different polarity values in each compartment 65, 70 will direct more than one type of molecule to the target electroporation site 55. Such molecular options can include different molecules crossing the membrane at the same or different times, including no transport at all, and will allow upon electroporation, the serial or parallel combination effect of the agent to be maximized.

A method in accordance with the present invention utilizes the combination of diffusion and electroporation. In a preferred embodiment, when a charge is placed on the top end cap 20 of the device 10, the charged entity within the vessel 15 will respond to that charge and move within the vessel accordingly. If the applied polarization charge on the top end cap 20 forces the charged entity within the vessel to migrate to the micro-porous membrane 30 at the bottom of the reservoir, this action will increase the chemical potential across the membrane and facilitate transport of the charged entity from the reservoir 15 through the porous membrane 30 and into the target tissue 55. After the transfer of the desired molecules into the target tissue 55, an appropriate electroporation protocol is initiated utilizing the electroporation applicators 35 imbedded in the bottom end cap 30 of the device 10. The electroporation protocol is effective in introducing the target molecule or charged entity into the cells of the target tissue 55.

The present invention provides for the delivery of a charged entity, typically a protein, plasmid or therapeutic drug, without the need of a needle or other intrusive delivery apparatus. The method and apparatus in accordance with the present invention is effective in adjusting the electrochemical potential of a target molecule thereby providing molecular transport of the target molecule into the tissue by a diffusive transport mechanism. With respect to a cell, organelle, or other subcellular compartment, the propensity of an electrically charged solute, to move across the micro-porous membrane is decided by the difference in its electrochemical potential on either side of the membrane. The subsequent application of the electroporation protocol to the electrode applicators in contact with the tissue surface, completes the treatment and delivers the target molecule into the interior of the cells of the target tissue.

It will be seen that the objects set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A molecular transport and electroporation apparatus comprising:
a reservoir having an electrically conductive top end cap and a micro-porous bottom end cap; and
the bottom end cap further comprising at least one electroporation applicator.

2. The apparatus of claim 1, further comprising an electrical insulator positioned to electrically isolate the conductive top end cap from the reservoir.

3. The apparatus of claim 1, further comprising a polarization supply voltage in circuit communication with the top end cap.

4. The apparatus of claim 1, wherein the micro-porous bottom end cap further comprises a micro-porous membrane; and
the at least one electroporation applicator is embedded in the micro-porous membrane of the bottom end cap.

5. The apparatus of claim 1, wherein the electroporation applicators are non-penetrating.

6. The apparatus of claim 1, wherein the at least one electroporation applicator further comprises a plurality of electroporation applicators.

7. The apparatus of claim 1, further comprising an electroporation supply voltage in circuit communication with the at least one electroporation applicator.

8. The apparatus of claim 1, further comprising a target molecule positioned within the reservoir.

9. The apparatus of claim 8, wherein the target molecule is a protein.

10. The apparatus of claim 8, wherein the target molecule is a plasmid.

11. The apparatus of claim 8, wherein the target molecule is a therapeutic drug.

12. The apparatus of claim 1, wherein the reservoir further comprises a plurality of isolated compartments.

13. The apparatus of claim 12, wherein each of the plurality of isolated compartments further comprises a target molecule positioned within each of the isolated compartments of the reservoir.

14. The apparatus of claim 12, wherein the top end cap of the reservoir further comprises a plurality of electrically isolated portions, each electrically isolated portion of the end cap associated with each of the plurality of isolated compartments.

15. A molecular transport and electroporation apparatus comprising:
a reservoir having an electrically conductive top end cap and a micro-porous bottom end cap;
the bottom end cap further comprising at least one electroporation applicator;
electrical insulator positioned to electrically isolate the conductive top end cap from the reservoir;
a polarization supply voltage in circuit communication with the top end cap;
at least one non-penetrating electroporation applicator integral to the bottom end cap;
an electroporation supply voltage in circuit communication with the at least one electroporation applicator.

16. The apparatus of claim 15, further comprising a target molecule positioned within the reservoir.

17. The apparatus of claim 15, wherein the target molecule is a protein.

18. The apparatus of claim 15, wherein the target molecule is a plasmid.

19. The apparatus of claim 15, wherein the target molecule is a therapeutic drug.

20. The apparatus of claim 15, wherein the reservoir further comprises a plurality of isolated compartments.

21. The apparatus of claim 20, wherein each of the plurality of isolator compartments further comprises a target molecule positioned within each of the isolated compartments of the reservoir.

22. The apparatus of claim 20, wherein the top end cap of the reservoir further comprises a plurality of electrically isolated portions, each electrically isolated portion of the end cap associated with each of the plurality of isolated compartments.

23. A method for molecular delivery and electroporation, the method comprising the steps of:
containing a target molecule within a reservoir;
contacting a micro-porous bottom end cap of the reservoir to a surface of a cellular tissue;
applying a polarization voltage to a conductive top end cap of the reservoir adapted to transport the target molecule through the micro-porous bottom end cap of the reservoir and diffuse the target molecule across the surface of the cellular tissue; and
applying an electroporation voltage to at least one electroporation applicator positioned on the micro-porous bottom end cap to introduce the target molecule into the interior of a cell of the cellular tissue.

24. The method of claim 23, wherein the step of applying a polarization voltage further comprises applying a polarization voltage following a predetermined protocol.

25. The method of claim 23, wherein the step of applying an electroporation voltage further comprises applying an electroporation voltage following a predetermined protocol.

26. The method of claim 23, further comprising the step of altering the electrochemical potential of the target molecule upon application of the polarization voltage to the conductive top end cap.

27. A method for molecular delivery and electroporation, the method comprising the steps of:
containing at least two distinct target molecules in a reservoir, each of the distinct target molecules contained within an isolated compartment of the reservoir;
applying a polarization voltage to at least an isolated portion of a conductive top end cap of the reservoir, each of the isolated portions of the top end cap associated with each of the isolated compartments of the reservoir, the application of the polarization voltage adapted to transport the target molecule through the micro-porous bottom end cap of the reservoir and diffuse the target molecule across the surface of the cellular tissue; and
applying an electroporation voltage to at least one electroporation applicator positioned on the micro-porous bottom end cap to introduce the target molecule into the interior of a cell of the cellular tissue.

28. The method of claim 27, wherein the step of applying a polarization voltage further comprises applying a polarization voltage following a predetermined protocol.

29. The method of claim 27, wherein the step of applying an electroporation voltage further comprises applying an electroporation voltage following a predetermined protocol.

30. The method of claim 27, further comprising the step of altering the electrochemical potential of the target molecule upon application of the polarization voltage to the conductive top end cap.
